(19) United States
(12) Patent Application Publication
(10) Pub. No.: US 2019/0134409 A1
(43) Pub. Date: May 9, 2019

(54) ELECTROSTATIC GENERATION OF ULTRASOUND POWER SIGNALS

(71) Applicant: QUALCOMM Incorporated, San Diego, CA (US)

(72) Inventors: William Henry VON NOVAK, III, San Diego, CA (US); Cody Burton WHEELAND, San Diego, CA (US); Linda Stacey IRISH, San Diego, CA (US)

(21) Appl. No.: 16/173,952

(22) Filed: Oct. 29, 2018

Related U.S. Application Data
(60) Provisional application No. 62/582,530, filed on Nov. 7, 2017.

(51) Int. Cl. A61N 1/378

(52) U.S. Cl. CPC A61N 1/3787

(57) ABSTRACT

Certain aspects of the present disclosure relate to systems and methods for using electrostatic deflection of tissue to generate ultrasound power signals, such as for powering medical implants. Certain aspects of the present disclosure provide a system for generating ultrasonic pressure waves. The system includes an electrode configured to be positioned near a tissue. The system further includes an AC signal generator coupled to the electrode, wherein the AC signal generator is configured to apply an AC signal to the electrode causing the tissue to vibrate to generate ultrasonic pressure waves directed to an ultrasonic power receiver implanted under the tissue.

### CROSS-REFERENCE TO RELATED APPLICATIONS

This application claims the benefit of U.S. Provisional Patent No. 62/582,530, filed Nov. 7, 2017. The content of the provisional application is hereby incorporated by reference in its entirety.

### TECHNICAL FIELD

The present disclosure relates generally to wireless power transfer, and in particular to systems and methods for using electrostatic deflection of tissue to generate ultrasound power signals, such as for powering medical implants.

### BACKGROUND

An increasing number and variety of electronic devices are powered via rechargeable batteries. Such devices include mobile phones, portable music players, laptop computers, tablet computers, computer peripheral devices, communication devices (e.g., Bluetooth® devices), digital cameras, hearing aids, medical implants, and the like. While battery technology has improved, battery-powered electronic devices increasingly require and consume greater amounts of power. As such, these devices constantly require recharging. Rechargeable devices are often charged via wired connections that require cables or other similar connectors that are physically connected to a power supply. Cables and similar connectors may sometimes be inconvenient or cumbersome and have other drawbacks. Wireless power transfer systems, for example, may allow users to charge and/or power electronic devices without physical, electrical connections, thus reducing the number of components required for operation of the electronic devices and simplifying the use of the electronic device.

For example, some battery powered devices, such as medical implants (e.g., pacemakers, neuromodulation devices, insulin pumps, etc.) may be located/positioned in areas where replacing the battery is not always feasible (e.g., in a body, such as, a human body). For example, to change a battery for a medical implant, surgery may need to be performed, which is risky. Accordingly, it may be safer to charge such devices wirelessly.

Further, some electronic devices may not be battery powered, but it still may be beneficial to utilize wireless power transfer to power such devices. In particular, the use of wireless power may eliminate the need for cords/cables to be attached to the electronic devices, which may be inconvenient and aesthetically displeasing.

Different electronic devices may have different shapes, sizes, and power requirements. There is flexibility in having different sizes and shapes in the components that make up a wireless power transmitter and/or a wireless power receiver in terms of industrial design and support for a wide range of devices

### SUMMARY

Certain aspects of the present disclosure provide a system for generating ultrasonic pressure waves. The system includes an electrode configured to be positioned near a tissue. The system further includes an AC signal generator coupled to the electrode, wherein the AC signal generator is configured to apply an AC signal to the electrode causing the tissue to vibrate to generate ultrasonic pressure waves directed to an ultrasonic power receiver implanted under the tissue.

Certain aspects of the disclosure provide a method for generating ultrasonic pressure waves. The method includes positioning an electrode near a tissue. The method further includes applying an AC signal to the electrode causing the tissue to vibrate to generate ultrasonic pressure waves directed to an ultrasonic power receiver implanted under the tissue.

Certain aspects of the disclosure provide a controller for generating ultrasonic pressure waves. The controller is configured to apply an AC signal to an electrode causing a tissue near the electrode to vibrate to generate ultrasonic pressure waves directed to an ultrasonic power receiver implanted under the tissue.

The following detailed description and accompanying drawings provide a better understanding of the nature and advantages of the present disclosure.

### BRIEF DESCRIPTION OF THE DRAWINGS

With respect to the discussion to follow and in particular to the drawings, it is stressed that the particulars shown represent examples for purposes of illustrative discussion, and are presented in the cause of providing a description of principles and conceptual aspects of the present disclosure. In this regard, no attempt is made to show implementation details beyond what is needed for a fundamental understanding of the present disclosure. The discussion to follow, in conjunction with the drawings, makes apparent to those of skill in the art how embodiments in accordance with the present disclosure may be practiced. In the accompanying drawings:

FIG. 1 is a functional block diagram of a wireless power transfer system, according to certain aspects of the present disclosure.

FIG. 2 illustrates an example of an electrostatic inducer system, according to certain aspects of the present disclosure.

FIG. 3 illustrates example operations for generating ultrasonic pressure waves, according to aspects of the present disclosure.

Drawing elements that are common among the following figures may be identified using the same reference numerals.

### DETAILED DESCRIPTION

As noted above, certain battery powered devices, such as medical implants (e.g., pacemakers, neuromodulation devices, insulin pumps, etc.) may be located/positioned in areas where replacing the battery is not always feasible (e.g., in a body, such as, a human body). For example, to change a battery for a medical implant, surgery may need to be performed, which is risky. Accordingly, it may be safer to charge such devices wirelessly (or power such devices wirelessly if they do not include a battery). For example, in some cases, ultrasonic energy transfer (e.g., through the use of pressure waves) may be used to wirelessly power a wireless power receiver, such as a medical implant.

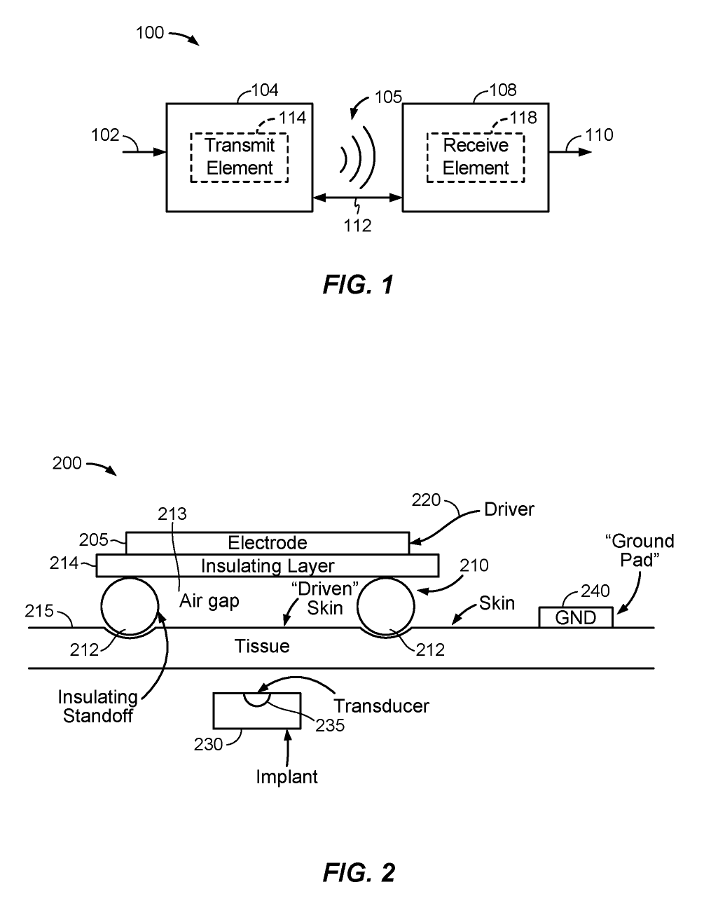

FIG. 1 is a functional block diagram of a wireless power transfer system 100, in accordance with certain aspects of the present disclosure. Input power 102 may be provided to a transmitter 104 from a power source (e.g., power supply, battery, mains electric power, etc., not shown in this figure) to generate ultrasonic pressure waves 105 (e.g., ultrasound power signals) for performing energy transfer. In particular, the transmitter 104 converts electrical energy in the form of input power 102 to mechanical energy in the form of ultrasonic pressure waves 105. The transmitter 104 may be configured to cause tissue (e.g., skin of a living being (e.g., animal, human, etc.)) to oscillate/vibrate to produce ultrasonic pressure waves 105 when exposed to electrostatic fields generated by conductive plates (e.g., electrodes) of the transmitter 104 based on input power 102. Example implementations of transmitter 104 are further discussed herein.

A receiver 108 (e.g., an ultrasonic transducer) may receive the ultrasonic pressure waves 105 and generate output power 110 for storing or consumption by a device (e.g., a medical implant, not shown in this figure) coupled to the output power 110. For example, the ultrasonic pressure waves 105 produced by transmitter 104 may be incident on an ultrasonic transducer of receiver 108. A conductive diaphragm of the ultrasonic transducer of receiver 108 may vibrate/oscillate due to the incident ultrasonic pressure waves 105 and generate an electric field through interaction with a conductive plate of the ultrasonic transducer. The transmitter 104 and the receiver 108 may be separated by a distance 112. The receiver 108 may include an ultrasonic pressure wave receiving element 118 for receiving the ultrasonic pressure waves and converting these waves into usable electric power.

For example, the ultrasonic pressure wave receiving element 118 may include an ultrasonic transducer that generates AC power from the interaction with ultrasonic pressure waves 105. The ultrasonic pressure wave receiving element 118 may further include a rectifier (e.g., bridge rectifier) configured to convert the AC power to DC power. In certain aspects, the voltage of the AC power generated at the ultrasonic transducer may be low, and therefore the rectifier may be a synchronous (e.g., switch based) rectifier. The ultrasonic pressure wave receiving element 118 may further include a capacitor or other filter to filter the DC power and supply the DC power for consumption by a device (e.g., a medical implant, not shown in this figure). In certain aspects, the ultrasonic pressure wave receiving element 118 may also include a DC-DC converter (e.g., boost converter and/or buck converter) to change the DC power voltage to a level suitable for the device.

Ultrasonic power may be useful for powering certain devices, such as medical implants implanted in a body of a living being. For example, ultrasonic power may have many benefits such as high power density (e.g., 200-7200 W/m² depending on area) and may have low loss within living tissue. Coupling ultrasonic energy to a body of a living being, however, may be difficult using traditional techniques. For example, ordinarily a large ultrasonic transducer is used to generate ultrasonic pressure waves, and the large ultrasonic transducer is coupled to the body using impedance-matching gel (e.g., impedance-matching gel is placed between the ultrasonic transducer and skin on the body) to cause the ultrasonic pressure waves to propagate into the body. Use of such gels and large transducers may be unwieldy, messy, and also inefficient.

Accordingly, certain aspects herein relate to an electrostatic inducer system (e.g., transmitter 104) configured to cause tissue of a living being to oscillate/vibrate to generate ultrasonic pressure waves. For example, the electrostatic inducer system may cause skin to vibrate at a frequency (e.g., 1-17 MHz) effective for ultrasonic energy transfer. The pressure signal caused by the vibration of the skin then propagates through the skin and other tissue (e.g., fat) of the body into an implant in the body. In certain aspects, the power transferred by such an electrostatic inducer system is proportional to the frequency of vibration of the tissue, such that a large deflection of the tissue is not needed to generate sufficient power for an implant.

FIG. 2 illustrates an example of an electrostatic inducer system 200, according to certain aspects. As shown, the system 200 includes an electrode 205 and one or more insulating layers 210. The electrode 205 is positioned above a surface of tissue 215 (e.g., skin) and insulated from the tissue 215 by the one or more insulating layers 210. In particular, the one or more insulating layers 210 are positioned between the electrode 205 and the tissue 215. The one or more insulating layers 210 may cause the electrode 205 to be positioned a short distance (e.g., 1-10 mm) away from the tissue 215 such that there is space between the electrode 205 and tissue 215 to prevent shock to the tissue 215 from electricity applied to the electrode 205.

The one or more insulating layers 210 may be made of one or more of foam, a soft material, or a gas (e.g., air, carbon dioxide, etc. in an air gap). For example, the one or more insulating layers 210 may include standoffs 212 (e.g., that contact the tissue 215 as shown) positioned such that there is an air gap 213 formed between the standoffs 212. The electrode 205 may then contact the standoffs 212 directly (not shown) or there may be one or more additional insulating layers 210 between standoffs 212 and electrode 205, such as insulating layer 214 as shown.

The electrode 205 is further coupled to a power source/AC signal generator 220 (e.g., including a high voltage driver) that supplies an AC signal (e.g., a high voltage AC signal) to energize the electrode 205. In certain aspects, the current output of the power source 220 is limited (e.g., on the order of mA) for safety by use of a low current driver to generate the AC signal. The AC signal energizing the electrode 205 causes alternating attraction and non-attraction between the tissue 215 and the electrode 205, resulting in vibration of the tissue 215 at a frequency of the AC signal (e.g., 1-17 MHz).

The vibration of the tissue 215 generates ultrasonic pressure waves which propagate through the tissue 215. A device 230 (e.g., medical implant) including an ultrasonic power receiver 235 (e.g., receiver 108) may be positioned below the tissue 215 and receive the ultrasonic pressure waves. The ultrasonic power receiver 235 may, based on the ultrasonic pressure waves, generate power for powering device 230 (e.g., charging a battery of device 230).

FIG. 2 further shows that a grounding pad 240 may also optionally be positioned on tissue 215 in certain aspects. The grounding pad 240 may be configured to ensure that the tissue 215 (e.g., and body including the tissue 215) remains at ground potential during operation of the electrostatic inducer system 200. However, such a grounding pad 240 may not be needed as at high frequencies, a body (e.g., human body) has low impedance to ground due to its large surface area and capacitive coupling to ground.

In certain aspects, electrostatic inducer system 200 may include a plurality of electrodes 205 (not shown) that are insulated from tissue 215 (e.g., by the same or different insulating layer(s) 210). For example, a plurality of electrodes 205 may be positioned near each other over tissue 215. In certain aspects, if a plurality of electrodes 205 are positioned near each other over tissue 215, they are driven with AC signals that are out of phase from one another. For example, if there are X electrodes 205 over tissue 215, each may be driven with an AC signal that is at 360/X degrees out of phase with the next closest AC signal. Accordingly, if there are two electrodes 205, they may be driven with AC signals 180 degrees out of phase from one another. If there are 4 electrodes 205, there may be 4 different AC signals to drive the 4 electrodes 205, with 90 degrees separation between the phases Utilizing different phases for the plurality of electrodes 205 may prevent degradation of the ground due to common mode currents.

In certain aspects, utilizing a plurality of electrodes 205 co-located to each other over tissue 215 may generate a potential difference between the electrodes 205 and tissue 215 without need for electrical contact with tissue 215/the body. In certain aspects, the plurality of electrodes 205 may include a center electrode 205 surrounded by an outer ring electrode 205. The center electrode 205 and outer ring electrode 205 may have similar surface area.

In certain aspects, the plurality of electrodes 205 may comprise an array of electrodes (e.g., formed as rows and columns). The plurality of electrodes 205 may be driven by AC signals with different phases to perform beamforming of the resulting ultrasonic pressure waves such as to steer ultrasonic pressure waves in the direction of the ultrasonic power receiver 235. The phase of the AC signals to cause the beamforming of ultrasonic pressure waves may controlled by a system controller (not shown), which may include one or more processors (not illustrated), such as a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein.

In certain aspects, the current of the electrostatic inducer system 200 is limited (e.g., to a maximum of 100 μA, to a maximum of 5 mA, etc.) so as to prevent dangerous currents from developing and injuring or shocking a user of the electrostatic inducer system 200. In certain aspects, the one or more insulating layers 210 help prevent contact between the tissue 215 and electrode 205, thereby further reducing the chance of shock. In certain aspects, electrode 205 includes a high resistivity material (e.g., a high density polyethylene), such as on the surface of electrode 205, so as to limit the current density if electrode 205 contacts tissue 215. In certain aspects, the high resistivity material may further be configured to generate a phase shift toward the outer edge of the transducer of the ultrasonic power receiver 235, which may allow for some phased array equivalent focusing/steering of ultrasonic pressure waves to ultrasonic power receiver 235, even if multiple electrodes 205 are not used for beamforming, or multiple different AC signals are not used to drive multiple electrodes 205 for beamforming. In certain aspects, electrode 205 may be formed of a flexible material and adhered to tissue 215 instead of positioned above the surface of tissue 215. In certain aspects, electrode 205 may be implanted in tissue 215.

In certain aspects, power source 220 is an isolated source that supplies the AC current to a first electrode 205, so that all of the return current is forced to return via AC coupling on a second electrode 205. This helps prevent current from flowing through the body over a large length.

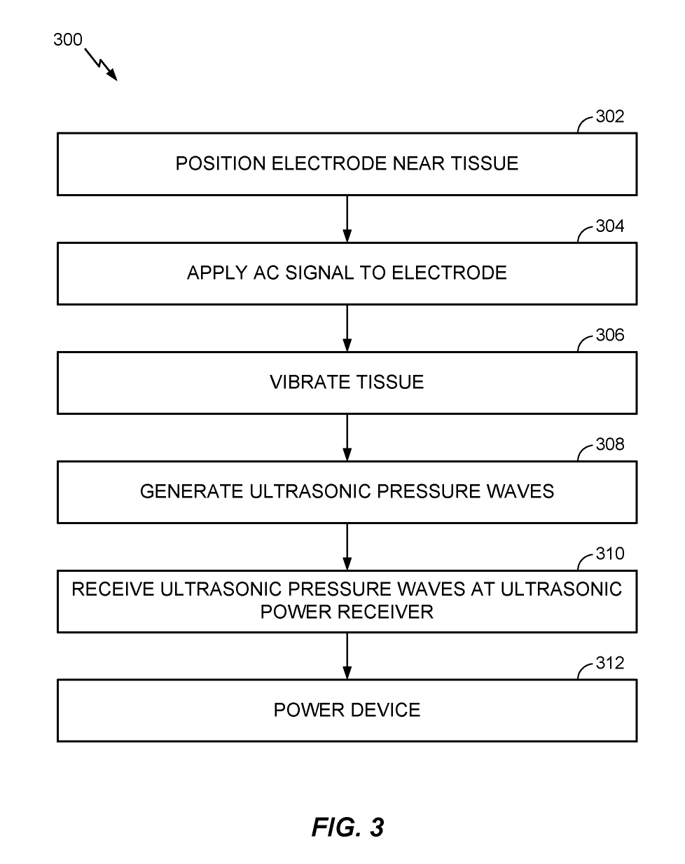

FIG. 3 illustrates example operations for generating ultrasonic pressure waves, according to aspects of the present disclosure. According to certain aspects, operations 300 may be performed, for example, by a wireless power transmitter, such as transmitter 104 or electrostatic inducer system 200.

Operations 300 begin at 302 by positioning an electrode near a tissue. For example, electrode 205 may be positioned above tissue 205, on tissue 205, or implanted in tissue 205 of an individual. Electrode 205 may be insulated from tissue 205 by one or more insulating layers 210. At 304, an AC signal is applied to the electrode. For example, power source 220 applies an AC signal at a first frequency to electrode 205. At 306, the tissue vibrates based on alternating attraction and non-attraction with electrode 205. For example, the electrode 205 driven by the AC signal attracts and does not attract the tissue at a frequency of the AC signal so the tissue vibrates at the frequency. At 308, the vibrations of the tissue forms ultrasonic pressure waves that propagate through tissue 205. For example, the ultrasonic pressure waves propagate through a body of the individual. At 310, the ultrasonic pressure waves are received at an ultrasonic power receiver (e.g., transducer). At 312, the ultrasonic power receiver powers a device using the received ultrasonic pressure waves.

The various operations of methods described above may be performed by any suitable means capable of performing the corresponding functions. The means may include various hardware and/or software component(s) and/or module(s), including, but not limited to a circuit, an application-specific integrated circuit (ASIC), or processor. Generally, where there are operations illustrated in figures, those operations may have corresponding counterpart means-plus-function components with similar numbering.

As used herein, the term “determining” encompasses a wide variety of actions. For example, “determining” may include calculating, computing, processing, deriving, investigating, looking up (e.g., looking up in a table, a database, or another data structure), ascertaining, and the like. Also, “determining” may include receiving (e.g., receiving information), accessing (e.g., accessing data in a memory), and the like. Also, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The various illustrative logical blocks, modules and circuits described in connection with the present disclosure may be implemented or performed with a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a wireless node. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement the signal processing functions of the physical (PHY) layer. In the case of a user terminal, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processing system may be configured as a general-purpose processing system with one or more microprocessors providing the processor functionality and external memory providing at least a portion of the machine-readable media, all linked together with other supporting circuitry through an external bus architecture. Alternatively, the processing system may be implemented with an ASIC with the processor, the bus interface, the user interface in the case of an access terminal), supporting circuitry, and at least a portion of the machine-readable media integrated into a single chip, or with one or more FPGAs, PLDs, controllers, state machines, gated logic, discrete hardware components, or any other suitable circuitry, or any combination of circuits that can perform the various functionality described throughout this disclosure. Those skilled in the art will recognize how best to implement the described functionality for the processing system depending on the particular application and the overall design constraints imposed on the overall system.

It is to be understood that the claims are not limited to the precise configuration and components illustrated above. Various modifications, changes and variations may be made in the arrangement, operation and details of the methods and apparatus described above without departing from the scope of the claims.

What is claimed is:

1. A system for generating ultrasonic pressure waves, the system comprising:
an electrode configured to be positioned near a tissue; and
an AC signal generator coupled to the electrode, wherein the AC signal generator is configured to apply an AC signal to the electrode causing the tissue to vibrate to generate ultrasonic pressure waves directed to an ultrasonic power receiver implanted under the tissue.

2. The system of claim 1, wherein the AC signal has a first frequency and the tissue vibrates at the first frequency.

3. The system of claim 1, wherein the AC signal generator is configured to generate the AC signal with a low current driver.

4. The system of claim 1, further comprising one or more insulating layers configured to be positioned between the electrode and the tissue.

5. The system of claim 1, further comprising a second electrode coupled to a second AC signal generator, wherein the second AC signal generator is configured to apply the second AC signal to the second electrode, wherein the AC signal and the second AC signal are out of phase.

6. The system of claim 1, further comprising a grounding pad configured to be positioned on the tissue.

7. The system of claim 1, wherein the electrode includes a resistive material on a surface of the electrode.

8. The system of claim 1, further comprising a plurality of electrodes; and a controller configured to drive the plurality of electrodes with different phases to steer the ultrasonic pressure waves to the ultrasonic power receiver.

9. A method for generating ultrasonic pressure waves, the method comprising:
positioning an electrode near a tissue; and
applying an AC signal to the electrode causing the tissue to vibrate to generate ultrasonic pressure waves directed to an ultrasonic power receiver implanted under the tissue.

10. The method of claim 9, wherein the AC signal has a first frequency and the tissue vibrates at the first frequency.

11. The method of claim 9, further comprising generating the AC signal with a low current driver.

12. The method of claim 9, further comprising insulating the electrode from the tissue.

13. The method of claim 9, further comprising applying a second AC signal to a second electrode positioned near the tissue, wherein the AC signal and the second AC signal are out of phase.

14. The method of claim 9, further comprising positioning a grounding pad on the tissue.

15. The method of claim 9, further comprising driving a plurality of electrodes with different phases to steer the ultrasonic pressure waves to the ultrasonic power receiver.

16. A controller for generating ultrasonic pressure waves, the controller being configured to:
apply an AC signal to an electrode causing a tissue near the electrode to vibrate to generate ultrasonic pressure waves directed to an ultrasonic power receiver implanted under the tissue.

17. The controller of claim 16, wherein the AC signal has a first frequency and the tissue vibrates at the first frequency.

18. The controller of claim 16, wherein the controller is further configured to apply a second AC signal to a second electrode positioned near the tissue, wherein the AC signal and the second AC signal are out of phase.

19. The controller of claim 16, wherein the controller is further configured to drive a plurality of electrodes with different phases to steer the ultrasonic pressure waves to the ultrasonic power receiver.

20. The controller of claim 16, wherein the controller comprises a low current driver.
